(19) United States
(12) Patent Application Publication
(10) Pub. No.: US 2021/0161846 A1
(43) Pub. Date: Jun. 3, 2021

(54) A STABLE PHARMACEUTICAL COMPOSITION COMPRISING PENICILLAMINE

(71) Applicant: KASHIV BIOSCIENCES, LLC, Bridgewater, NJ (US)

(72) Inventors: Parva Yogeshchandra PUROHIT, Ahmedabad (IN); Paras Rasiklal VASANANI, Ahmedabad (IN); Vikas Maheshbhai AGRAWAL, Ahmedabad (IN); Jitendra Shantilal PATEL, Mahesana (IN); Nisha Ganapatbhai PATEL, Ahmedabad (IN)

(21) Appl. No.: 16/770,926

(22) PCT Filed: Dec. 6, 2018
(86) PCT No.: PCT/IB18/59701

§ 371 Date: Jun. 8, 2020

(30) Foreign Application Priority Data
Dec. 9, 2017 (IN) IN201721044297

(51) Int. Cl. A61K 31/198; A61K 9/00; A61K 9/08; A61K 47/10; A61K 47/12; A61K 47/38; A61K 47/22; A61K 47/02; A61K 47/14; A61K 47/18; A61K 9/16

(52) U.S. Cl. CPC A61K 31/198; A61K 9/0053; A61K 9/08; A61K 47/10; A61K 47/12; A61K 47/38; A61K 9/1652; A61K 47/02; A61K 47/14; A61K 47/183; A61K 9/1623; A61K 9/1611; A61K 9/1617; A61K 47/22

(57) ABSTRACT

The present invention relates to a pharmaceutical composition comprising penicillamine which remains stable for a longer period of time at room temperature. The present invention specifically addresses problem associated with impurity generation during a penicillamine composition preparation and bad odour of penicillamine active ingredient. The pharmaceutical composition of present invention comprises penicillamine and one or more pharmaceutically acceptable excipients wherein the said composition is present in liquid dosage form. Moreover, the present invention composition provides patient compliance aspect in the treatment of Wilson's disease, Rheumatoid Arthritis and Cystinuria.

### TECHNICAL FIELD

The present invention relates to a pharmaceutical composition comprising penicillamine which remains stable for a longer period of time at room temperature. The present invention specifically addresses problem associated with impurity generation during a penicillamine composition preparation and bad odour of penicillamine active ingredient. The pharmaceutical composition of present invention comprises penicillamine and one or more pharmaceutically acceptable excipients wherein the said composition present in liquid dosage form. Moreover, the present invention composition provides patient compliance aspect in the treatment of Wilson's disease, Rheumatoid Arthritis and Cystinuria.

### BACKGROUND ART

Penicillamine is a chelating agent used for the treatment of wilson's disease, rheumatoid arthritis and cystinuria. The chemical name of penicillamine is 3-mercapto-D-valine and its chemical structure is represented by following FIG. 1.

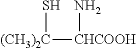

### FIG. 1: Structural Formula of Penicillamine

D-penicillamine is commercially available under the brand names of Cuprimine and Depen®. Cuprimine is available as 125 milligram (mg) and 250 milligram (mg) capsules and Depen® is available as 250 milligram (mg) scored tablets.

GB 1424432 relates to a process of preparing fully synthetic d-penicillamine and discloses that medicament can be administered in the form of tablets, capsules, pills, dragees, suppositories, ointments, jellies, powders, liquids, dusting powders or aerosols. It further discloses suitable liquids include oily or aqueous solutions or suspensions, emulsions, injectable aqueous or oily solutions or suspensions.

Penicillamine is a sulfur containing drug having an unpleasant odour. The unpleasant odour of penicillamine renders the medication unpalatable and may lead to missed doses. Further, penicillamine absorption is significantly reduced by the presence of calcium so it is not given with dairy products or food.

Wilson's disease is a rare autosomal recessive inherited disease that causes excess copper accumulation primarily in the liver, brain, kidneys, and cornea; it affects about one in 30,000 people worldwide. The liver of a person inflicted with Wilson's disease does not release copper into bile as it should; accordingly, copper builds up in the liver injuring the liver tissue.

The required dose for the treatment of Wilson's disease, Rheumatoid arthritis and Cystinuria is too high and hence, patients need to takemultiple doses at a time. The dose of penicillamine in the treatment of Wilson's disease varies from 750 mg to 1500 mg per day and if required need to increase up to 2000 mg per day. The dose of penicillamine in the treatment of Cystinuria is 2000 mg/day for adult patients varying with a range of 1000 mg to 4000 mg per day and for pediatric patients dosage can be based on 30 mg/kg/day. The maintenance dose of penicillamine in the treatment of Rheumatoid arthritis varies from 500 mg to 750 mg per day.

Currently, penicillamine available dosage form includes tablets or capsules which are the most convenient method of drug administration, however due to higher dose in treatment, patients need to take about 2 to 16 tablets or capsules at a time depending on the severity of disease which is cumbersome and inconvenient to patients especially for neonates, infants and elders. Further, due to unpleasant odour of the active compound, it creates resistance into patients for following frequent adopted practice of crushing and dispersing the tablets or opened capsules content into a flavoured vehicle for easy administration of medicaments. So, this leads to occurrence of medication errors in patients who are in chronic stage of therapy.

US 20070134277 describe the pharmaceutical formulation for sulphur-containing drugs in liquid dosage form or a dry powder dosage form for reconstitution in water. The said patent application emphasizes on taste masking of the pungent sulphur odour of d-penicillamine and also discloses about poor stability of d-penicillamine in liquid dosage forms. The '277 patent application also discloses d-penicillamine impurity which is formed as a dimer d-penicillamine disulphide into solution and should be considered as a degradation product.

From the above disclosure of prior arts, it appears that there remains an unmet need to prepare stable composition of penicillamine which obviates problems associated with penicillamine active ingredient such as stability, bad odour, and desirable con-centration of drug which provides a patient compliance treatment. The above prior arts disclose general aspects of preparing penicillamine solution dosage form with impurity formation into formulation such as dimer called as penicillamine disulphide, but none of these focus on the long-term stability aspect of penicillamine active ingredient into solution form. Further, none of the prior arts disclose a method of controlling impurity into the penicillamine solution during a longer period of time.

Drummer et al. publication “Reversibility of disulphide formation comparison of chemical and enzyme-mediated reduction of penicillamine and captopril disulfides”University of Melbourne, Vol. 36, No. 8, pp 1197-1201 discloses penicillamine disulphide dimer which accumulates into body. It discloses that disulphides are not readily excreted by the kidney and may cause problems during chronic therapy.

The inventor of the present invention have addressed the above problems associated with penicillamine composition and have successfully discovered that it is possible to prepare a stable composition of penicillamine which provides long term stability. Further, the inventors have addressed the long unmet need of patients associated with currently available therapy of penicillamine into the market till date and provide a compliance based solution in terms of dosage form administration aspects especially into infants, neonates and elders patients. Also, the inventors have addressed the bad odour problem associated with penicillamine active ingredient by incorporating taste masking excipients which do not have any interaction into the composition.

### SUMMARY OF INVENTION

The present invention relates to a pharmaceutical composition comprising penicillamine, which remains stable for longer period of time at room temperature.

The present invention relates to a stable pharmaceutical composition of present invention comprising penicillamine and a one or more pharmaceutically acceptable excipients. Specifically, the present invention composition is provided into liquid dosage form which possesses pH in range from 2 to 6. The present invention specifically relates to a novel process of preparing stable pharmaceutical composition of penicillamine in which impurity profile is well-controlled and complies with the limits of ICH standard.

Further, the present invention relates to a stable pharmaceutical composition of penicillamine which overcomes bad odour problems of active ingredient by incorporating a suitable taste masking agent into the composition.

Moreover, the present invention addresses patient compliance treatment aspects of penicillamine therapy and can be used in the treatment of Wilson's disease, Rheumatoid Arthritis and Cystinuria.

### DESCRIPTION OF EMBODIMENTS

The present invention relates to a stable pharmaceutical composition comprising penicillamine and a novel process of preparing the pharmaceutical composition.

Specifically, the present invention relates to a pharmaceutical composition comprising penicillamine which remains stable for long period of time at room temperature.

The present invention relates to a stable pharmaceutical composition comprising penicillamine, and a one or more pharmaceutically acceptable excipients wherein the said composition is present in liquid dosage form.

Specifically, the present invention composition is provided into liquid dosage form which possesses pH in range from 2 to 6.

The inventors of the present invention have addressed the problems associated with solution dosage forms of penicillamine and have successfully discovered a stable pharmaceutical composition of penicillamine having a long-term stability at room temperature. The present invention emphasize on problems associated with penicillamine active ingredient in solution form which generates dimer called as penicillamine disulphide and this dimer amount increases over a period of time during storage. As per reported literature, in solution, penicillamine degrades slowly by first order kinetics. Further, this penicillamine disulphide accumulates into patients who are taking penicillamine on routine basis and therefore creates a problem during a long-term therapy.

The pharmaceutical composition of present invention addressed problems associated with currently marketed tablets or capsules dosage forms of penicillamine in patients and provides compliance based solution in terms of dosage administration aspects. Specifically, elders or pediatric patient including neonates, infants etc., who are unable to swallow the medication or have difficulty in swallowing medication and need to crush tablets and dispersed into water or disperse capsule content into water, the present invention composition provides advantageous solution to these problems.

Further, the inventors have addressed problems with penicillamine active ingredient which is a sulfur containing drug having an unpleasant odour. The unpleasant odour of penicillamine leads to reduction of patient compliance aspect and may lead to missed doses. Hence, the present invention provides a solution by formulating composition in such a manner which solves problem of bad odour of active ingredient and provides better patient compliance.

The inventors have defined term “stable” used here in the context of formulating a stable pharmaceutical composition in which total impurities level of both specified and unspecified complies as per ICH standards.

The term “impurities” includes specified and unspecified substances. The specified impurities include dimer such as penicillamine disulphides, penicillamine trisulphides etc.

The term “penicillamine” is used to refer to isomers of penicillamine, pharmaceutically acceptable salts, esters and prodrugs thereof, the active metabolites of penicillamine, polymorphs, solvates, hydrates, and combinations thereof. The isomers of penicillamine include the d-isomer and the 1-isomer out of which preferably the d-isomer of penicillamine is present in the invention.

The term “about” refers to any value which lies within the range defined by the present inventors that varies upto±10% of the claimed value.

The term “liquid dosage form” used herein is defined as the dosage forms which include solution, suspension, syrup, elixir, dry powder dosage forms for reconstitution in vehicle or alike thereof.

According to present invention, the pharmaceutical composition is stable for atleast six months, atleast twelve months, atleast eighteen months and atleast twenty-four months having disulphide less than about 5%, preferably less than about 4% and more preferably less than about 3% and trisulphide less than about 0.5%, preferably less than about 0.3% and more preferably less than about 0.15%. The pharmaceutical composition provides palatable odour and taste.

In first aspect of the present invention, there is provided a stable pharmaceutical composition comprising penicillamine and at least one pharmaceutically acceptable excipient selected from group consisting of a stabilizing agent, pH adjusting agent, flavouring agent, sweetener, preservative and optionally dispersants, buffering agent and solvent.

In second aspect of the present invention, a stable pharmaceutical composition is present in form of solution, suspension, syrup or a dry powder dosage form for reconstitution in water or alike.

In third aspect of the present invention, a stable pharmaceutical composition of penicillamine is present in liquid dosage form having a pH in range from about 2 to about 6, preferably from about 2 to about 4.

According to one embodiment of the present invention, a stable composition comprises penicillamine in an amount ranging from about 10 mg/ml to about 1000 mg/ml, preferably in an amount from about 50 mg/ml to about 1000 mg/ml, more preferably from about 100 mg/ml to about 500 mg/ml.

According to further embodiment of the present invention, a stable composition comprises penicillamine in an amount ranging from about 10% to about 50% w/v, preferably about 20% to about 40% w/v.

According to another embodiment of the present invention, a stable liquid composition comprises of penicillamine and a one or more pharmaceutically acceptable excipients. Examples of non-limiting pharmaceutically acceptable excipients includes, stabilizing agents, pH adjusting agents, buffering agents, flavouring agents, sweeteners, preservatives, solvents/co-solvents, dispersants, diluents, vehicles or alike thereof.

According to another embodiment of the present invention, a stabilizing agent is an agent which provides stability to the penicillamine liquid composition. Examples of stabilizing agents include but not limited to antioxidants, reducing agents, chelating agents or alike thereof. The stabilizing agent is present in an amount ranging from about 0.005% to about 15% w/v.

Examples of antioxidants include but not limited to a butylated hydroxyanisole (BHA), butylated hydroxytoluene (BHT), tetra butyl hydroquinone, gallic acid, propyl gallate, α-tocopherol, ascorbic acid, citric acid, L-cysteine, thioglycolic acid and alike thereof. The antioxidant is present in an amount ranging from about 0.02% to about 5% w/v.

Examples of reducing agents include but not limited to, ascorbyl palmitate, monothioglycerol, sodium bisulfite, sodium metabisulfite, sodium sulfite and alike thereof. The reducing agent is present in an amount ranging from about 0.02% to about 5% w/v.

Examples of chelating agents include but not limited to a citric acid, trisodium citrate, disodium edetate, sodium diethyldithiocarbamate, fumaric acid, malic acid, phosphoric acid, tartaric acid, gallic acid, glutamic acid, oxalic acid and alike thereof. The chelating agent is present in an amount ranging from about 0.02% to about 5% w/v.

The pH adjusting agents used in the pharmaceutical composition of present invention are selected from inorganic acids, organic acids and alike thereof. Inorganic acids include hydrochloric acid, sulphuric acid, phosphoric acid and alike thereof. Organic acids include citric acid, tartaric acid, malic acid, fumaric acid, succinic acid and alike thereof. Preferably, pH adjusting agent in the present invention composition is hydrochloric acid and tartaric acid. The pH adjusting agent is present in an amount sufficient to adjust pH of the liquid dosage form.

The flavouring agents used in the pharmaceutical composition of present invention are selected from the group consisting of a grape, apple, anise, apricot, blackberry, blueberry, cherry syrup, cherry mint, cherry-black, cherry-red, cranberry, fennel, ginger, guava, liquorice, lime, lemon, maple, mint, orange, passion fruit, peach, pineapple, plum, prune, peppermint, raspberry, rose, strawberry, spearmint, wild cherry syrup and alike thereof. The flavouring agent is present in an amount ranging from about 0.1% to about 10% w/v.

The sweeteners used in the pharmaceutical composition of present invention are selected from the group consisting of maltitol, xylose, ribulose, glucose (dextrose), mannose, galactose, fructose (levulose), sucrose (table sugar), maltose, invert sugar (amixture of fructose and glucose derived from sucrose), partially hydrolyzed starch, corn syrup solids, dihydrochalcones, monellin, steviosides, glycyrrhizin or glycyrrhizin derivatives, and sugar alcohols such as sorbitol, mannitol, xylitol, hydrogenated starch hydrolysates and soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one2,2-dioxide (Acesulfame-K) and a com-bination mixtures thereof. The sweetener is present in an amount ranging from about 0.5% to about 10% by w/v.

The preservatives used in the pharmaceutical composition of present invention are selected from the group consisting of a benzalkonium chloride, sodium benzoate, benzyl alcohol, bronopol, cetrimide, cetylpyridinium chloride, chlorhexidine, chlorobutanol, chlorocresol, chloroxylenol, cresol, ethyl alcohol, glycerine, hexetidine, imidurea, phenol, phenoxyethanol, phenylethyl alcohol, phenylmercuric nitrate, thimerosal, butylparaben, methylparaben, ethylparaben, propylparaben, benzoic acid, potassium sorbate, sodium propionate, sorbic acid and alike thereof. The preservative is present in an amount ranging from about 0.1% to about 5% w/v.

The solvents/co-solvents used in the pharmaceutical composition of present invention are selected from the group consisting of a propylene glycol, glycerin, water soluble polyethylene glycol (PEG) polymers, propylene glycol and alike thereof. The solvent/co-solvent is present in an amount ranging from about 1% to about 30% w/v, preferably from about 5% to about 20% w/v.

The dispersants used in the pharmaceutical composition of present invention are selected from the group consisting of a carbopol, methylcellulose, hydroxyl-propylmethyl cellulose (HPMC), hydroxyethyl cellulose, hydroxypropyl cellulose, sodium carboxymethyl cellulose (CMC), polyvinyl alcohol, polyvinylpyrrolidone, polyacrylates, polyacrylamide, dextran, gellan gum, poloxamer, calcium poly-carbophil, cellulose acetate phthalate, sodium hyaluronate, hyaluronic acid, alginate, chitosan and alike thereof. The dispersant is present in an amount ranging from about 01% to 10% w/v.

The buffering agents used in the pharmaceutical composition of the present invention to maintain the pH of the liquid dosage form after adjusting pH with pH adjusting agents. Suitable buffering agents include citrate, lactate, phosphate, tromethamine, glycine, borate, acetate salts and alike thereof. The buffering agent is present in an amount sufficient to maintain pH of the liquid dosage form.

The vehicle used in the pharmaceutical composition is selected from water, water-ethanol mixture and alike thereof. The vehicle used in an amount to maintain final volume of the composition.

According to present invention, a stable oral liquid composition possesses a viscosity which is appropriate for administration via an intraoral. The viscosity of the composition must be low enough to allow such flow.

Another aspect of the invention is to provide an oral, pharmaceutical liquid composition comprising penicillamine, which is bioequivalent to the commercially available penicillamine tablet & capsule formulations, marketed under the brand name “Depen” and “Cuprimine”.

Preferably, the stable pharmaceutical composition is present in form of solution or a dry powder dosage form for reconstitution.

The stable oral solution comprises about 20% to about 40% w/v penicillamine, about 0.02% to about 15% w/v stabilizing agent, pH adjusting agent, about 0.1% to about 10% w/v flavouring agent, about 0.5% to about 10% w/v sweetener, about 0.1% to about 5% w/v preservative, about 0% to about 10% w/v dispersants and buffering agent if require.

The dry powder composition comprises penicillamine, diluent and stabilizing agent.

In further embodiment, the dry powder composition comprises about 20% to about 40% w/w penicillamine, about 20% to about 80% w/w diluent, about 0.02% to about 15% w/w stabilizing agent, about 0.1% to about 10% w/w flavouring agent, about 0.5% to about 10% w/w sweetener and about 0.1% to about 5% w/w preservative and 0% to about 5% glidant.

In some embodiments, when the dry powder composition is reconstituted into vehicle and, the liquid is homogenous and stable for at least 4 weeks, atleast 8 weeks and atleast 12 weeks at ambient or refrigerated conditions. The vehicle is aqueous vehicle having a pH in range from about 2 to about 6; preferably from about 2 to about 4. The dry powder composition is stable for at least six months, at least twelve months, at least eighteen months and atleast twenty-four months at ambient or refrigerated conditions.

Examples of diluent include but not limited to sugar like monosaccharides such as glucose, fructose, galactose; disaccharides such as sucrose, maltose, lactose; sugar alcohol such as sorbitol, mannitol, isomalt and polysaccharides such as maltodextrin or celluloses derivatives like microcrystalline cellulose or powdered celluloses and alike thereof. The diluent is present in an amount ranging from about 20% to 80% w/w.

Examples of binder include but not limited to cellulose derivatives like hydroxypropyl methyl cellulose, ethyl cellulose, methyl cellulose, polyvinyl alcohol and like thereof. The binder is present in an amount ranging from about 0% to 15% w/w.

Examples of glidant include but not limited to talc, stearates, magnesium carbonate, magnesium oxide, calcium silicate, and colloidal silicon dioxide. The glidant is present in an amount ranging from about 0% to 5% w/w.

The vehicle used for reconstitution of dry powder composition is aqueous vehicle having pH between about 2 to about 6, preferably about 2 to about 4.

The dry powder composition of the present invention can be produced using the con-ventional manufacturing procedures such as homogenisation, sieving and milling. A portion of the ingredients may be pre-granulated, or granulated ingredients can be used to improve powder flowability.

The composition of present invention can be stored in any container suitable for maintaining stability. Preferably, the composition can be stored in amber colour container. The composition can be dispensed, for example, by loading into an automated medication dispensing system, by extraction with a syringe, or by pouring the composition directly into a device (e.g., a syringe or machine) for administration to a patient. Additionally, the composition can be dispensed by metered dose dispenser. Further, the pharmaceutical composition can be stored under refrigerated conditions (2° C. to 8 OC), at room temperature or at well-below room temperature conditions for a longer period of time for atleast six months, atleast 12 months, atleast 18 months and atleast twenty-four months.

The dry powder composition can be stored in the sachet packaging or plastic bottle packaging with separate bottle of reconstitution vehicle.

In one embodiment, the liquid composition does not experience substantial penicillamine degradation for a period of at least about 1 year when stored under refrigerated, at room temperature and well below room temperature conditions. In another embodiment, the composition does not experience substantial penicillamine degradation for a period of at least about two years when stored under refrigerated, at room temperature and well below room temperature conditions.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### EXAMPLES

### Example 1: Composition for Penicillamine Oral Solution

TABLE 1

| Ingredients | Range (% w/v) |
|---|---|
| Penicillamine | 20 |
| BHA | 0.07 |
| BHT | 0.07 |
| Citric acid | 1 |
| Sodium benzoate | 0.5 |
| Sodium carboxymethyl cellulose | 0-5 |
| Saccharin sodium | 0.5 |
| Wild cherry flavour | 0.1 |
| Hydrochloric acid | to pH around 2 |
| Purified water | q.s. upto 100 ml |

### Process for Preparation:

1) Charge the batching vessel with purified water. With continuous moderate agitation, add hydrochloric acid to achieve pH around 3 to 4.

2) With continuous moderate agitation, add penicillamine to the batching vessel from Step 1 and mix thoroughly.

3) With continuous moderate agitation, add BHA, BHT, citric acid and sodium benzoate.

4) With continuous moderate agitation, add Saccharin sodium and wild cherry flavour flavour into above solution. Also, add carboxymethyl cellulose to achieve proper viscosity, if required.

5) Finally, if require to adjust pH around 2, then add hydrochloric acid.

6) Filter the solution from Step 5 through a stainless steel screen.

7) Store the solution from Step 6 in a tank.

### Example 2: Composition for Penicillamine Oral Solution

TABLE 2

| Ingredients | Range (% w/v) |
|---|---|
| Penicillamine | 20 |
| Ascorbic acid | 1 |
| Methylparaben | 0.2 |
| Propylparaben | 0.02 |
| Sodium carboxymethyl cellulose | 0-5 |
| Aspartame | 1 |
| Orange flavour | 0.5 |
| Hydrochloric acid | to pH around 2 |
| Purified water | q.s. upto 100 ml |

### Process for Preparation:

1) Charge the batching vessel with purified water. With continuous moderate agitation, add hydrochloric acid to achieve pH around 3 to 4.

2) With continuous moderate agitation, add penicillamine to the batching vessel from Step 1 and mix thoroughly.

3) With continuous moderate agitation, add ascorbic acid, methylparaben and propylparaben.

4) With continuous moderate agitation, add aspartame and orange flavour into above solution. Also, add carboxymethyl cellulose to achieve proper viscosity, if required.

5) Finally, if require to adjust pH around 2, then add hydrochloric acid.

6) Filter the solution from Step 5 through a stainless steel screen.

7) Store the solution from Step 6 in a tank.

### Example 3: Composition for Penicillamine Oral Solution

TABLE 3

| Ingredients | Range (% w/v) |
|---|---|
| Penicillamine | 20 |
| Ascorbic acid | 0.6 |
| Sodium metabisulphite | 0.8 |
| Methylparaben | 0.2 |
| Propylparaben | 0.02 |
| Sodium carboxymethyl cellulose | 0-5 |
| Aspartame | 1 |
| Grape flavour | 0.5 |
| Hydrochloric acid | to pH around 2 |
| Purified water | q.s. upto 100 ml |

### Process for Preparation:

1) Charge the batching vessel with purified water. With continuous moderate agitation, add hydrochloric acid to achieve pH around 3 to 4.

2) With continuous moderate agitation, add penicillamine to the batching vessel from Step 1 and mix thoroughly.

3) With continuous moderate agitation, add ascorbic acid, sodium metabisulphite, methylparaben and propylparaben.

4) With continuous moderate agitation, add aspartame and grape flavour into above solution. Also, add carboxymethyl cellulose to achieve proper viscosity, if required.

5) Finally, if require to adjust pH around 2, then add hydrochloric acid.

6) Filter the solution from Step 5 through a stainless steel screen.

7) Store the solution from Step 6 in a tank.

### Example 4: Composition for Penicillamine Oral Solution

TABLE 4

| Ingredients | Range (% w/v) |
|---|---|
| Penicillamine | 20 |
| Sodium metabisulphite | 1 |
| Methylparaben | 0.2 |
| Propylparaben | 0.02 |
| Sodium carboxymethyl cellulose | 0-5 |
| Saccharin sodium | 0.5 |
| Strawberry flavour | 0.2 |
| Hydrochloric acid | to pH around 3 |
| Purified water | q.s. upto 100 ml |

### Process for Preparation:

1) Charge the batching vessel with purified water. With continuous moderate agitation, add hydrochloric acid to achieve pH around 3.

2) With continuous moderate agitation, add penicillamine to the batching vessel from Step 1 and mix thoroughly.

3) With continuous moderate agitation, add sodium metabisulphite, methylparaben and propylparaben.

4) With continuous moderate agitation, add saccharin sodium and strawberry flavour into above solution. Also, add carboxymethyl cellulose to achieve proper viscosity, if required.

5) Finally, if require to adjust pH around 3, then add hydrochloric acid.

6) Filter the solution from Step 5 through a stainless steel screen.

7) Store the solution from Step 6 in a tank.

Stability Data of Penicillamine Oral Solution at Ambient Condition:

TABLE 5

| Example | Impurity | Initial | 1 Month | 3 Month | 6 Month |
|---|---|---|---|---|---|
| 1 | Disulphide | 0.3 | 1 | 1.9 | 2.8 |
| | Trisulphide | 0.04 | 0.07 | 0.1 | 0.14 |
| 2 | Disulphide | 0.3 | 0.7 | 1.2 | 1.9 |
| | Trisulphide | 0.03 | 0.05 | 0.07 | 0.1 |
| 3 | Disulphide | 0.3 | 0.6 | 1.0 | 1.7 |
| | Trisulphide | 0.03 | 0.04 | 0.05 | 0.09 |
| 4 | Disulphide | 0.3 | 0.9 | 1.7 | 2.7 |
| | Trisulphide | 0.04 | 0.06 | 0.09 | 0.12 |

### Example 5—Composition for Penicillamine Dry Powder for Reconstitution

TABLE 6

| Ingredients | Range (% w/w) |
|---|---|
| Penicillamine | 25 |
| Mannitol | 66.6 |
| Ascorbic acid | 1 |
| HPMC 15 cPs | 5 |
| Saccharin sodium | 0.5 |
| Strawberry Flavour | 0.2 |
| Sodium carboxymethyl cellulose | 1 |
| Sodium benzoate | 0.5 |
| colloidal silicone dioxide | 0.2 |

### Process for Preparation:

1) Sift Penicillamine, mannitol, ascorbic acid, strawberry flavour and saccharin sodium through appropriate sieve and load in the fluid-bed processor. Allow the excipients to mix for 10 mins.

2) Maintain the temperature of blend at 40° C. for preheating.

3) Dissolve HPMC in water with continuous stirring and continue the same during the process.

4) Spray the binder solution over the drug excipients blend at appropriate spray rate maintaining the blend temperature at 40° C.-42° C. throughout the spray process.

5) Once the spraying is complete, allow drying of the formed granules at 40° C. for 10-15 mins.

6) Sift the formed granules through sieve.

7) Sift sodium carboxymethyl cellulose, colloidal silicon dioxide and sodium benzoate through #60 sieve.

8) Blend the formed granules with the excipients sifted in Step-7 for atleast 5 mins.

9) The final blend at the end of step-8 is then filled in suitable dispensing package as final dosage form.

### Example 6—Composition for Penicillamine Dry Powder for Reconstitution

TABLE 7

| Ingredients | Range (% w/w) |
|---|---|
| Penicillamine | 20 |
| Lactose | 72 |
| HPMC 15 cPs | 5 |
| Sodium metabisulphite | 1 |
| Methylparaben | 0.1 |
| Propylparaben | 0.01 |
| Aspartame | 1 |
| Sodium carboxymethyl cellulose | 0.2 |
| Grape flavour | 0.5 |
| colloidal silicone dioxide | 0.2 |

### Process for Preparation:

1) Sift Penicillamine, lactose, sodium metabisulphite and aspartame through appropriate sieve and load in the fluid-bed processor. Allow the excipients to mix for 10 mins.

2) Maintain the temperature of blend at 40° C. for preheating.

3) Dissolve HPMC in water with continuous stirring and continue the same during the process

4) Spray the binder solution over the drug excipients blend at appropriate spray rate maintaining the blend temperature at 40° C.-42° C. throughout the spray process.

5) Once the spraying is complete, allow drying of the formed granules at 40° C. for 10-15 mins.

6) Sift the formed granules through sieve.

7) Sift sodium carboxymethyl cellulose, colloidal silicon dioxide, methyl paraben and propyl paraben and grape flavor through #60 sieve.

8) Blend the formed granules with the excipients sifted in Step-7 for atleast 5 mins.

9) The final blend at the end of step-8 is then filled in suitable dispensing package as final dosage form.

Stability Data of Penicillamine Dry Powder Composition after Reconstitution at Ambient Condition:

To reconstitute the dry powder composition in aqueous vehicle having pH between 2 to 4 to obtain a solution containing about 200 mg/mL of d-penicillamine. The solutions obtained from the formulation examples 5 and 6 were placed at ambient condition and samples were drawn and evaluated after 7 days, 15 days and 30 days for the presence of penicillamine disulphide and penicillamine trisulphide.

TABLE 8

| | Impurity | | | | |
|---|---|---|---|---|---|
| Example | (% w/w) | Initial | 7 days | 15 days | 30 days |
| 5 | Disulphide | 0.3 | 0.5 | 0.7 | 0.8 |
| | Trisulphide | 0.04 | 0.05 | 0.06 | 0.08 |
| 6 | Disulphide | 0.3 | 0.6 | 0.7 | 0.9 |
| | Trisulphide | 0.03 | 0.05 | 0.07 | 0.09 |

1.-21. (canceled)

22. A stable composition for oral administration comprising a penicillamine and at least one pharmaceutically acceptable excipient; wherein the composition is stable for at least 6 months having impurity level of disulphide & trisulphide in an amount of less than about 5%.

23. The composition according to claim 22, wherein penicillamine is present in amount from about 10 to about 50% w/v.

24. The composition according to claim 22, wherein the pH of the composition is about 2 to about 6.

25. The composition according to claim 22 having impurities level of disulphide in an amount of less than about 4% and impurities level of trisulphide in an amount of less than about 0.5%.

26. The composition according to claim 22 having impurities level of disulphide in an amount of less than about 3% and impurities level of trisulphide is in an amount of less than about 0.15%.

27. The composition according to claim 22, wherein the composition is present in form of solution, suspension, syrup, elixir, dry powder dosage forms for reconstitution.

28. The composition according to claim 22, wherein the composition is stable for at least six months at ambient or refrigerated conditions.

29. The composition according to claim 22, wherein the pharmaceutically acceptable excipient is selected from the group consisting of stabilizing agent, flavouring agent, sweetening agent, pH adjusting agent, diluent, glidant preservative, dispersant, buffering agent, water or mixture thereof.

30. The composition according to claim 29, wherein the stabilizing agent is selected from the group consisting of anti-oxidant, reducing agent, or mixture thereof; and wherein the stabilizing agent is present in amount from about 0.01% to about 15% w/v.

31. The composition according to claim 30, wherein the anti-oxidant is selected from group consisting of butylated hydroxyanisole, butylated hydroxytoluene, tetra butyl hydroquinone, gallic acid, propyl gallate, α-tocopherol, ascorbic acid, citric acid, malic acid, tartaric acid, L-cysteine, thioglycolic acid or mixture thereof; and wherein the anti-oxidant is present in an amount from about 0.02% to about 5% w/v.

32. The composition according to claim 30, wherein the reducing agent is selected from group consisting of ascorbyl palmitate, monothioglycerol, sodium bisulphite, sodium metabisulphite, sodium sulphite or mixture thereof; and wherein the reducing agent is present in amount from about 0.01% to about 5% w/v.

33. The composition according to claim 27, wherein the composition is present in form of solution.

34. The composition according to claim 33, wherein the composition comprising about 20% to about 40% w/v penicillamine, about 0.02% to about 15% w/v stabilizing agent, about 0.1% to about 10% w/v flavouring agent, about 0.5% to about 10% w/v sweetener, about 0.1% to about 5% w/v preservative, pH adjusting agent, 0% to about 10% w/v dispersants.

35. The composition according to claim 27, wherein the composition is present in form of dry powder dosage forms for reconstitution.

36. The composition according to claim 35, wherein the composition comprising about 20 to about 40% w/w penicillamine, about 20 to about 80% w/w diluent, 0.005% to about 15% w/w stabilizing agent, about 0.1% to about 10% w/w flavouring agent, about 0.5% to about 10% w/w sweetener, about 0.1% to about 5% w/w preservative and 0% to about 2% w/w glidant.
